(19) United States
(12) Patent Application Publication
(10) Pub. No.: US 2016/0186159 A1
(43) Pub. Date: Jun. 30, 2016

(54) SOLID TEXTILE DETERGENT WITH IMPROVED PROTEASE PERFORMANCE

(71) Applicant: Henkel AG & Co. KGaA, Duesseldorf (DE)

(72) Inventors: Timothy O'Connell, Duesseldorf (DE); Susanne Tondera, Duesseldorf (DE); Hendrik Hellmuth, Duesseldorf (DE); Thomas Weber, Dormagen (DE); Nina Mussmann, Willich (DE)

(21) Appl. No.: 15/064,808

(22) Filed: Mar. 9, 2016

Related U.S. Application Data
(63) Continuation of application No. PCT/EP2014/065470, filed on Jul. 18, 2014.

(30) Foreign Application Priority Data
Sep. 12, 2013 (DE) 10 2013 218 253.8

(51) Int. Cl. C12N 9/54; C11D 3/386

(52) U.S. Cl. CPC C12N 9/54; C11D 3/38609

(57) ABSTRACT

The aim of the invention is to improve the detergent power of solid textile detergents having a pH value ranging from 4 to under 10 in a solution containing 1 wt. % in demineralized water at 20° C. Said aim is achieved by using a protease which comprises an amino acid sequence that is at least 80% identical to the amino acid sequence set forth in SEQ ID NO. 1 and contains, at position 99 of SEQ ID NO. 1, the amino acid glutamic acid (E) or aspartic acid (D), or the amino acid asparagine (N) or glutamine (Q), or the amino acid alanine (A) or glycine (G) or serine (S).

### FIELD OF THE INVENTION

The present invention generally relates to solid textile detergents, and more particularly relates to solid enzyme-containing textile detergents containing certain proteases, and furthermore proposes methods in which such agents are used. The invention further relates to the use of certain proteases in solid textile detergents.

### BACKGROUND OF THE INVENTION

Proteases of the subtilisin type are preferred for detergents and cleaning agents. The proteases that are used in the detergents or cleaning agents known from the prior art either originally come from microorganisms, for example the genus Bacillus, Streptomyces, Humicola or Pseudomonas, and/or are produced according to biotechnology methods that are known per se using suitable microorganisms, for example using transgenic expression hosts of the Bacillus genus or using filamentous fungi.

The international patent application WO 95/23221 discloses proteases or protease variants of the subtilisin type from Bacillus lentus DSM 5483, which are suitable for use in detergents or cleaning agents. These proteases also include one that may carry an amino acid exchange R99E, A, S or G. The detergents can be solid or liquid. This document, however, does not clearly and unambiguously reveal that a solid textile detergent is involved which at position 99 contains the amino acid glutamic acid (E) or aspartic acid (D), or the amino acid asparagine (N) or glutamine (Q), or the amino acid alanine (A) or glycine (G) or serine (S), and has a certain pH value.

The described proteases are known as ingredients of liquid detergents or cleaning agents. For example, advantageous liquid detergents or cleaning agents that contain one of the desciibed proteases in combination with cellulases are described in the international patent application WO 2012/080201. Liquid detergents or cleaning agents that contain one of the described proteases in combination with amylases are described in WO 2012/080202.

Advantages of the described proteases over conventional proteases of the prior art in solid automatic dishwashing agents are disclosed in the German patent application DE 102012215107.9. Solid automatic dishwashing agents generally have high pH values of 10 or more. Surprisingly, no advantage of the described proteases in solid textile detergents having a similar pH profile as in automatic solid dishwashing agents has been found to this day.

High pH values in the washing liquor, and thus also high alkali values in the detergents, are known to contribute to the washing performance. With respect to textile care, however, they can have negative effects, so that lower pH values are desirable with respect to the care and protection of textiles.

The object was therefore to improve the washing performance of solid textile detergents having a lower pH value in a 1 wt. % solution in deionized water at 20° C.

Surprisingly, it was found that, in solid textile detergents having a lower pH value in a 1 wt. % solution in deionized water at 20° C., the above-mentioned proteases exhibit advantages over the previous proteases, which were previously used in solid textile detergents.

Furthermore, other desirable features and characteristics of the present invention will become apparent from the subsequent detailed description of the invention and the appended claims, taken in conjunction with this background of the invention.

### BRIEF SUMMARY OF THE INVENTION

A solid textile detergent, comprising: (a1) a protease including an amino acid sequence that is at least 80% identical to the amino acid sequence indicated in SEQ ID NO. 1 and contains the amino acid glutamic acid (E) or aspartic acid (D) at position 99 in the count according to SEQ ID NO. 1; or (a2) a protease including an amino acid sequence that is at least 80% identical to the amino acid sequence indicated in SEQ ID NO. 1 and contains the amino acid asparagine (N) or glutamine (Q) at position 99 in the count according to SEQ ID NO. 1; or (a3) a protease including an amino acid sequence that is at least 80% identical to the amino acid sequence indicated in SEQ ID NO. 1 and contains the amino acid alanine (A) or glycine (G) or serine (S) at position 99 in the count according to SEQ ID NO. 1, wherein the solid textile detergent in a 1 wt. % solution in deionized water at 20° C. has a pH value in a range from 4.0 to less than 10.

Use of a protease, (al.) which includes an amino acid sequence that is at least 80% identical to the amino acid sequence indicated in SEQ ID NO. 1 and contains the amino acid glutamic acid (E) or aspartic acid (D) at position 99 in the count according to SEQ ID NO. 1; or (a2) which includes an amino acid sequence that is at least 80% identical to the amino acid sequence indicated in SEQ ID NO. 1 and contains the amino acid asparagine (N) or glutamine (Q) at position 99 in the count according to SEQ ID NO. 1; or (a3) which includes an amino acid sequence that is at least 80% identical to the amino acid sequence provided in SEQ ID NO. 1 and the amino acid alanine (A) or glycine (G) or serine (S) at position 99 in the count according to SEQ ID NO. 1, so as to provide a proteolytic activity in a solid textile detergent that in a 1 wt. % solution in deionized water at 20° C. has a pH value in the range from 4 to less than 10.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The presen nventio first relates to a solid textile detergent, comprising:
(a1) a protease including an amino acid sequence that is at least 80% identical to the amino acid sequence indicated in SEQ ID NO. 1 and contains the amino acid glutamic acid (E) or aspartic acid (D) at position 99 in the count according to SEQ ID NO. 1; or
(a2) a protease including an amino acid sequence that is at least 80% identical to the amino acid sequence indicated in SEQ ID NO. 1 and contains the amino acid asparagine (N) or glutamine (Q) at position 99 in the count according to SEQ ID NO, 1; or
(a3) a protease including an amino acid sequence that is at least 80% identical to the amino acid sequence provided in SEQ ID NO. 1 and contains the amino acid alanine (A) or glycine (G) or serine (S) at position 99 in the count according to SEQ ID NO. 1, wherein the solid textile detergent in a 1 wt. % solution in deionized water at 20° C. has a pH value in the range from 4.0 to less than 10.

Solid textile detergents according to the invention in particular exhibit performance advantages over other solid textile detergents even when the solid textile detergents contain at least one additional enzyme of the same or of another type, which is to say, for example, amylase, cellulase, lipase, mannanase or pectinase, wherein the list of the further enzymes is not exhaustive. It is therefore preferred that the solid textile detergents according to the invention contain at least one additional enzyme of the same type (which is to say, a further protease) or of another type.

The agents according to the invention in particular exhibit an advantage over conventional solid textile detergents even when they do not contain any bleach, and in particular no inorganic or organic peroxide bleach source.

In an advantageous embodiment of the invention, the solid textile detergents have a pH value (1 wt. % solution in deionized water at 20° C.) of 6.5 to 9.5, and in particular of 7.0 to 9.0.

An agent according to the invention in particular exhibits an advantageous cleaning performance on protease-sensitive soiling. Such cleaning performance with respect to at least one protease-sensitive soil occurs in particular also at low temperatures, for example at washing temperatures of 10° C. to 50° C., preferably of 10° C. to 40° , or of 20° C. to 40° C. Such an agent thus allows the satisfactory or improved removal of at least one, preferably of multiple protease-sensitive soils on textiles. Cleaning performance within the scope of the invention shall be understood to mean the lightening performance on one or multiple soils, in particular laundry soils. Examples of such soils include blood on cotton or chocolate-milk/soot on cotton, cocoa on cotton, or porridge on cotton.

The protease present in a solid textile detergent according to the invention includes an amino acid sequence that is at least 80% identical to the amino acid sequence provided in SEQ ID NO. 1 and, at position 99 in the count according to SEQ ID NO. 1, contains the amino acid glutamic acid (E) or aspartic acid (D), or the amino acid asparagine (N) or glutamine (Q), or the amino acid alanine (A) or glycine (G) or serine (S). The amino acid sequence is increasingly preferably at least 81%, 82%, 83%, 84%, 85%, 86%, 87%, 88%, 89%, 90%, 91%, 92%, 93%, 94%, 95%, 96%, 97%, 98%, and most particularly preferably 99% identical to the amino acid sequence indicated in SEQ ID NO. 1, SEQ ID NO. 1 is the sequence of the mature alkaline protease from Bacillus lentus DSM 5483, which is disclosed in the international patent application WO 92/21760 and to the disclosure of which explicit reference is hereby made.

A protease present in a solid textile detergent according to the invention exhibits proteolytic activity, which is to say it is capable of hydrolyzing peptide bonds of a polypeptide or protein. It is thus an enzyme which catalyzes the hydrolysis of peptide bonds and is thereby able to cleave peptides or proteins. It is in particular a subtilase, and particularly preferably a subtilisin.

In a further embodiment of the invention, the solid textile detergent is characterized in that the protease, in the count according to SEQ ID NO. 1, furthermore includes at least one of the following amino acids:

(a) threonine at position 3 (3T);

(b) isoleucine at position 4 (4I);

(c) alanine, threonine or arginine at position 61 (61A, 61T or 61R);

(d) aspartic acid or glutamic acid at position 154 (154D or 154E);

(e) proline at position 188 (188P);

methionine at position 193 (193M);

(g) isoleucine at position 199 (1991);

(h) aspartic acid, glutamic acid or glycine at position 211 (211D, 211E or 211G),

(i) combinations of amino acids (a) to (h).

In addition to one of the described amino acids at position 99, the protease thus includes one or more of the above-mentioned amino acids at the respective positions. These amino acids can induce fiirther advantageous properties and/or further enhance existing properties. In particular, the above-mentioned amino acids induce an increase in the proteolytic activity and/or the stability of the protease in a solid textile detergent or in the washing liquor formed by this detergent. By adding such a protease to a solid textile detergent, a particularly storage-stable solid textile detergent is thus likewise obtained.

The amino acid positions are defined by an alignment of the amino acid sequence of the protease to be used with the amino acid sequence of the protease from Bacillus lentus, as it is indicated in SEQ ID NO. 1. Since the protease from Bacillus lentus in the prior art represents an important reference molecule for describing proteases and amino acid changes, it is advantageous to reference the count of the protease from Bacillus lentus (SEQ ID NO. 1) in the assignment of the amino acid positions. The count is furthermore dependent on the mature protein. This assignment should in particular also be used when the amino acid sequence of the protease to be used comprises a higher number of amino acid residues than the protease from Bacillus lentus according to SEQ ID NO. 1. Proceeding from the described positions in the amino acid sequence of the protease from Bacillus lentus, the amino acid positions in a protease to be used according to the invention are those which are assigned to these very positions in an alignment.

Particularly advantageous positions in addition to position 99 are thus positions 3, 4, 61, 154, 188, 193, 199 and 211, to be assigned in an alignment with SEQ ID NO. 1 and hence in the count according to SEQ ID NO. 1. At the described positions, the following amino acid residues are present in the wild type molecule of the protease from Bacillus lentus: S3, V4, G61, S154, A188, V193, V199, and L211. The amino acids 3T, 41, 61A, 154D, 154E, 211D, 211G and 211E are particularly preferred, provided that the corresponding positions are not already taken up in a protease to be used according to the invention by one of these preferred amino acids. The exchanges 3T and 4I, for example, have a stabilizing effect on the molecule, resulting in an improvement in the storage stability and cleaning performance of the protease and hence in an improved cleaning performance of a solid textile detergent containing the protease. If one or more of the above-mentioned amino acids are present at the respective position, further sequence variations from SEQ ID NO. 1 result, in addition to position 99, since SEQ ID NO. 1 has a different amino acid at the respective position. Different maximum identity values that a protease to be used according to the invention can have in relation to SEQ ID NO. 1 therefore result as a function of the number of sequence variations that are present from SEQ ID NO. 1, even if the protease were to correspond to SEQ ID NO. 1 in all other amino acids. This fact must be taken into consideration in the individual case for every possible combination of the proposed amino acids and is moreover also dependent on the length of the amino acid sequence of the protease. For example, the maximum identity with one, two, three, four, five, six, seven, eight or nine sequence changes is 99.63%, 99.26%, 98.88%, 98.51%, 98.14%, 97.77%, 97.40%, 97.03% or 96.65%, respectively, for a 269 amino acid-long amino acid sequence, or 99.64%, 99.27%, 98.91%, 98.55%, 98.18%, 97.82%, 97.45%, 97.09% or 96.73%, respectively, for a 275 amino acid-long amino acid sequence.

The identity of nucleic acid or amino acid sequences is determined by way of a sequence comparison. Such a comparison is carried out by assigning similar sequences in the nucleotide sequences or amino acid sequences to one another. This sequence comparison is preferably carried out based on the BLAST algorithm that is established in the prior art and customarily used (see, for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pgs. 3389-3402) and is essentially carried out by assigning similar successions of nucleotides or amino acids in the nucleic acid or amino acid sequences to each other. A tabular assignment of the particular positions is referred to as alignment. Another algorithm available in the prior art is the FASTA algorithm. Sequence comparisons (alignments), in particular multiple sequence comparisons, are usually created using computer programs. For example, the Clustal series (see, for example, Chenna et al, (2003): Multiple sequence alignment with the Clustal series of programs. Nucleic Acid Research 31, 3497-3500), T-Coffee (see, for example. Notredame et al. (2000): T-Coffee: A novel method for multiple sequence alignments. J. Mol. Biol. 302, 205-217) or programs that are based on these programs or algorithms are frequently used. Within the scope of the present invention, sequence comparisons and alignments are preferably created with the computer program Vector NIT® Suite 10.3 (Invitrogen Corporation, 1600 Faraday Avenue, Carlsbad, Calif., USA) using the predefined default parameters.

Such a comparison allows information to be provided about the similarity of the compared sequences among each other. It is customarily indicated in percent identity, which is to say the share of identical nucleotides or amino acid residues at the same positions or at positions corresponding to each other in an alignment. The broader concept of homology, in the case of amino acid sequences, takes conservative amino acid substitutions into consideration, which is to say amino acids having similar properties, since these generally carry out similar activities or functions within the protein. The similarity of the compared sequences may thus also be indicated in percent homology or percent similarity. Identity and/or homology information can be provided for entire polypeptides or genes, or only for individual regions. Homologous or identical regions of different nucleic acid or amino acid sequences are therefore defined by agreement in the sequences. They oft have identical or similar functions. They may be small and comprise only few nucleotides or amino acids. Such small regions often carry out functions that are essential for the overall activity of the protein. It may therefore be useful to relate sequence agreements only to individual, optionally small regions. Unless indicated otherwise, however, identity or homology information in the present application refers to the total length of the respective indicated nucleic acid or amino acid sequence.

In a further embodiment of the invention, the solid textile detergent is characterized in that the protease includes an amino acid sequence that is identical to the amino acid sequence indicated in SEQ ID NO. 1 as indicated above and that is obtained, or obtainable, from a protease according to SEQ 1D NO. 1 by one or more conservative amino acid substitutions, wherein the protease at position 99 still includes one of the amino acids intended for this position, as described above. The expression “conservative amino acid substitution” refers to the exchange (substitution) of one amino acid residue for another amino acid residue, wherein this exchange does not result in a change in the polarity or charge at the position of the exchanged amino acid, such as the exchange of a nonpolar amino acid residue for another non-polar amino acid residue. Conservative amino acid substitutions within the scope of the invention include, for example: G=A=S, I=V=L=M, D=E, N=Q, K=R, Y=F, S=T, G=A,=I=V=L=M=Y=F=W=P=S=T.

Numerous proteases, and in particular subtilisins, are formed as what are known as pre-proteins, which is to say together with a pro-peptide and a signal peptide, wherein the function of the signal peptide is usually to ensure the release of the protease from the cell producing it into the periplasm or the medium surrounding the cell, and the pro-peptide is usually necessary for the correct folding of the protease. The signal peptide and the pro-peptide are generally the N-terminal part of the pre-protein. The signal peptide is cleaved from the remainder of the protease under natural conditions by way of a signal peptidase. The correct final folding of the protease then takes place, supported by the pro-peptide. The protease is then in the active form thereof and cleaves the pro-peptide itself. Following the cleavage of the pro-peptide, the then mature protease, in particular subtilisin, carries out the catalytic activity thereof without the originally present N-terminal amino acids. For technical applications in general, and within the scope of the invention in particular, the mature proteases, which is to say the enzymes processed after production of the same, are preferred over the pre-proteins. The proteases can moreover be modified by the cells producing them following the production of the polypeptide chain, for example by the attachment of sugar molecules, formylations, aminations, and the like. Such modifications are post-translational modifications and may, but do not have to, influence the function of the protease.

An agent according to the invention increasingly preferably contains the protease in an amount of 1×10⁻⁸ to 5 wt. %, 0.0001 to 1 wt. %, 0.0005 to 0.5 wt. %, 0.001 to 0.1 wt. %, each based on active protein. The protein concentration can be determined using known methods, such as the BCA method (bicinchoninic acid; 2,2′-bichinolyl-4,4′-dicarbonic acid) or the biuret method (A, G. Gomall, C. S. Bardawill and M. M. David, J. Biol. Chem., 177 (1948), pgs. 751-766). The active protein concentration was determined in this regard by a titration of the active centers using a suitable irreversible inhibitor (for proteases, for example, phenylmethylsulfonyl fluoride (PMSF)) and determination of the residual activity (see N I. Bender et al., J. Am. Chem. Soc. 88, 24 (1966), pgs. 5890-5913).

The protease and/or at least one additional enzyme of the same or another type can be adsorbed on carrier substances and/or be embedded in coating substances to protect them against premature inactivation. In the washing liquor, which is to say under usage conditions, the enzyme is then released and can develop its catalytic effect. The protease of the type mentioned above thus formulated can be present in the solid textile detergent in amounts of 0.05 to 5 wt. %, preferably in amounts of 0.05 to 2 wt. %. Preferred formulation forms comprise 0.05 to 15 wt. %, and in particular up to 10 wt. %, active protein of the described protease.

In addition to this protease, the solid textile detergents can contain further enzymes and preferred solid textile detergents preferably comprise at least one further enzyme, the at least one further enzyme being a further protease, an amylase, a cellulase, hemicellulase, mannanase, tannase, xylanase, xanthanase, xyloglucanase, β-glucosidase, pectinase, carrageenase, perhydrolase, oxidase, oxidoreductase or a lipase, and the mixtures thereof Further enzymes are advantageously each present in the agent in an amount from 1×10⁻⁸ to 5 wt. %, based on active protein. Each further enzyme is increasingly preferably present in the solid textile detergents in an amount from 1×10⁻⁷ to 3 wt. %, from 0.00001 to 1 wt. %, from 0.00005 to 0.5 wt. %, from 0.0001 to 0.1 wt. %, and particularly preferably from 0.0001 to 0.05 wt. %, based on active protein. The enzymes particularly preferably demonstrate synergistic cleaning performance with respect to certain soils or stains, which is to say the enzymes present in the agent composition support one another in the cleaning performance thereof. Such synergy most particularly preferably exists between the protease contained according to the invention and a further enzyme, including in particular between the described protease and an amylase and/or a lipase and/or a mannanase and/or a cellulase and/or a pectinase. Synergistic effects can occur not only between different enzymes, but also between one or more enzymes and other ingredients of the agent according to the invention. Preferred solid textile detergents thus comprise at least one protease and at least one amylase. In a further preferred embodiment of the invention, solid textile detergents comprise at least one protease and at least one cellulase. In a further preferred embodiment, solid textile detergents comprise at least one protease and at least one lipase. Solid textile detergents comprising 3 to 10 different enzymes are particularly preferred, wherein solid textile detergents comprising 3 to 10 different enzymes can be particularly preferred with respect to the cleaning performance regarding a very large range of stains.

The solid textile detergents can be present as powdery to granular solids, but also in compacted or post-compacted form. In principle, all known ingredients customary in such agents can be present. The solid textile detergents according to the invention can in particular contain builder substances, surfactants, the enzymes already mentioned above, enzyme stabilizers, sequestering agents, electrolytes, pH regulators and further auxiliary agents, such as optical brighteners, graying inhibitors, color transfer inhibitors, foam regulators, and dyes and odorants. While bleach-free agents, and in particular peroxide-free agents, are particularly preferred, bleaching agents such as peroxygen compounds and peroxygen activators, but also metal-containing or metal-free bleach catalysts, are not precluded in general.

Possible suitable organic or inorganic peroxygen compounds include organic peroxy acids or peracid salts of organic acids, such as phthalimidopercaproic acid, perbenzoic acid or salts of diperdodecanoid diacid, in particular, however, inorganic peroxygen compounds such as hydrogen peroxide, and inorganic salts giving off hydrogen peroxide under the cleaning conditions, such as perborate, percarbonate and/or persilicate, and hydrogen peroxide clathrates, such as 11202 urea adducts. Hydrogen peroxide may also be created by way of an enzymatic system, which is to say an oxidase and the substrate thereof. To the extent that solid peroxygen compounds are to be used, these may be used in the form of powders or granules, which may also be coated in the manner known per se. The peroxygen compounds can be added to the washing or cleaning lye as they are or in the form of agents containing the same, which in principle can contain all customary detergent, cleaning agent or disinfecting agent components. The use of alkali percarbonate or alkali perborate monohydrate is particularly preferred. If a solid textile detergent according to the invention contains peroxygen compounds, these are preferably present in amounts of 0.1 wt. % to 20 wt. %.

Suitable bleach activators are compounds that carry O- and/or N-acyl groups and/or optionally substituted benzoyl groups. Polyacylated alkylenediamines, in particular tetra acetyl ethylene diamine (TAED), acylated glycolurils, in particular tetraacetyl glycoluril (TAGU), acylated triazine derivatives, in particular 1,5-diacetyl-2,4-dioxohexahydro-1,3,5-triazine (DADHT), N-acylimides, in particular N-nonanoyl succinimide (NOSI), acylated phenolsulfonates or phenolcarboxylates or the sulfonic or carboxylic acids of the same, in particular nonanoyl or iso-nonanoyl or lauroyl oxybenzene sulfonate (NOBS or iso-NOBS or LOBS), 4-(2-decanoyloxyethoxycarbonyloxy)benzene sulfonate (DECOBS) or decanoyloxybenzoate (DOBA), acylated polyhydric alcohols, in particular triacetin, ethylene glycol diacetate and 2,5-diacetoxy-2,5-dihydrofuran, and acetylated sorbitol and mannitol and the mixtures thereof (SORMAN), acylated sugar derivatives, in particular penta-acetyl glucose (PAG), penta-acetyl fructose, tetra-acetyl xylose and octa-acetyl lactose, acetylated, optionally N-alkylated glucamine and gluconolactone, N-acylated lactams, for example N-benzoyl caprolactam, nitriles, from which perimidic acids form, in particular aminoacetonitrile derivatives having a quaternized nitrogen atom, and/or oxygen-transferring sulfonimines and/or acylhydrazones are preferred. These bleach activators can preferably be present in the agents up to 10 wt. %.

The agents according to the invention can contain one or more surfactants, wherein in particular anionic surfactants, nonionic surfactants, and the mixtures thereof may be used. Suitable nonionic surfactants are in particular alkylglycosides and ethoxylation and/or propoxylation products of alkylglycosides or linear or branched alcohols, each having 8 to approximately 18 carbon atoms in the alkyl part and 3 to 20, preferably 4 to 10 alkyl ether groups. Furthermore, corresponding ethoxylation and/or propoxylation products of N-alkyl amines, vicinal diols, fatty acid esters and fatty acid amides, which with respect to the alkyl part correspond to the described long-chain alcohol derivatives, and of alkyl phenols having 5 to 12 carbon atoms in the alkyl group may be used.

Suitable anionic surfactants are in particular soaps and those that contain sulfate or sulfonate groups including preferably alkali ions as cations. Soaps that can be used are preferably the alkali salts of the saturated or unsaturated fatty acids having 12 to 18 carbon atoms. Such fatty acids can also be used in not fully neutralized form. The surfactants of the sulfate type that may be used include the salts of the sulfuric acid semiesters of fatty alcohols having 12 to 18 carbon atoms and the sulfation products of the described nonionic surfactants having a low degree of ethoxylation. The surfactants of the sulfonate type that may be used include linear alkylbenzene sulfonates having 9 to 14 carbon atoms in the alkyl part, alkane sulfonates having 12 to 18 carbon atoms, and olefin sulfonates having 12 to 18 carbon atoms, which are created during the reaction of corresponding monoolefins with sulfur trioxide, and alpha-sulfo fatty acid esters, which are created during the sulfonation of fatty acid methyl or ethyl esters.

Such surfactants are present in the solid textile detergents according to the invention in proportions of preferably 5 wt. % to 50 wt. %, and in particular of 8 wt. % to 30 wt. %.

An agent according to the invention preferably contains at least one water-soluble and/or water-insoluble, organic and/or inorganic builder. The water-soluble organic builder substances include polycarboxylic acids, in particular citric acid, saccharic acids and carboxymethyl inulins, monomeric and polymeric aminopolycarboxylic acids, in particular glycine diacetic acid, methylglycine diacetic acid, nitrilotriacetic acid, iminodisuccinates such as ethylenediamine-N,N′-disuccinic acid and hydroxyiminodisuccinates, ethylenediaminetetraacetic acid and polyaspartic acid, polyphosphonic acids, in particular aminotris(methylenephosphonic acid), ethylenediaminetetrakis(methylenephosphonic acid), lysine tetra(methylenephosphonic acid) and 1-hydroxyethane-1,1 -diphosphonic acid, polymeric hydroxy compounds such as dextrin and polymeric (poly-)carboxylic acids, in particular polycarboxylates accessible by oxidation of polysaccharides, polymeric acrylic acids, methacrylic acids, maleic acids, and mixed polymers of the same, which may also have small proportions of polymerizable substances having no carboxylic acid functionality polymerized into the same. The relative average molecular mass (here and hereafter: weight average) of the homopolymers of unsaturated carboxylic acids is generally between 5,000 g/mol and 200,000 g/mol, that of the copolymers is between 2,000 g/mol and 200,000 g/mol, preferably 50,000 g/mol to 120,000 g/mol, each based on free acid. A particularly preferred acrylic acid-maleic acid copolymer has a relative average molecular mass of 50,000 g/mol to 100,000 g/mol. Suitable, albeit less preferred compounds of this class are copolymers of acrylic acid or methacrylic acid with vinyl ethers, such as vinyl methyl ethers, vinyl esters, ethylene, propylene and styrene, in which the proportion of the acid is at least 50 wt. %. It is also possible to use terpolymers containing two unsaturated acids and/or the salts thereof as the monomers, and vinyl alcohol and/or a vinyl alcohol derivative or a carbohydrate as the third monomer, as water-soluble organic builder substances. The first acid monomer or the salt thereof is derived from a monoethylenically unsaturated C₃-C₈ carboxylic acid and preferably from a C₃-C₄ monocarboxylic acid, in particular from (meth)acrylic acid. The second acid monomer or the salt thereof can be a derivative of a C₄-C₈ dicarboxylic acid, wherein maleic acid is particularly preferred. The third monomeric unit is formed in this case by vinyl alcohol and/or preferably an esterified vinyl alcohol. In particular vinyl alcohol derivatives are preferred which represent an ester of short-chain carboxylic acids, for example of C₁-C₄ carboxylic acids, with vinyl alcohol. Preferred polymers contain 60 wt. % to 95 wt. %, in particular 70 wt. % to 90 wt. %, (meth)acrylic acid or (meth)acrylate, particularly preferably acrylic acid or acrylate, and maleic acid or maleinate, and 5 wt. % to 40 wt. %, preferably 10 wt. % to 30 wt. %, vinyl alcohol and/or vinyl acetate. Most particularly preferred are polymers in which the weight ratio of (meth)acrylic acid or (meth)acrylate to maleic acid or maleinate ranges between 1:1 and 4:1, preferably between 2:1 and 3:1, and in particular 2:1 and 2.5:1. Both the amounts and the weight ratios are based on the acids. The second acid monomer or the salt thereof can also be a derivative of an aflyl sulfonic acid, which at the 2-position is substituted with an alkyl group, preferably a C₁-C₄ alkyl group, or an aromatic group, which is preferably derived from benzene or benzene derivatives. Preferred terpolymers contain 40 wt. % to 60 wt. %, in particular 45 wt. % to 55 wt. %, (meth)acrylic acid or (meth)acrylate, particularly preferably acrylic acid or acrylate, 10 wt. % to 30 wt. %, preferably 15 wt.'%© to 25 wt. %, methallyl sulfonic acid or methallyl sulfonate, and, as the third monomer, 15 wt. % to 40 wt. %, preferably 20 wt. % to 40 wt. % of a carbohydrate. This carbohydrate can be a mono-, di-, oligo- or polysaccharide, for example, wherein mono-, di- or oligosaccharides are preferred. Sucrose is particularly preferred. As a result of the use of the third monomer, predetermined breaking points are presumably introduced into the polymer, which are responsible for the good biodegradability of the polymer. These terpolymers generally have a relative average molecular mass between 1,000 g/mol and 200,000 g/mol, preferably between 200 g/mol and 50,000 g/mol. Further preferred copolymers are those that contain acrolein and acrylic acid/acrylic acid salts or vinylacetate as monomers. All described acids are generally used in the form of the water-soluble salts thereof, in particular the alkali salts thereof.

Such organic builder substances can he present in amounts of up to 40 wt. %, in particular up to 25 wt. %, and preferably from 1 wt. % to 8 wt. %, if desired.

Water-insoluble inorganic builder materials that are used are in particular crystalline or amorphous, water-dispersible alkali aluminosilicates, preferably in amounts not above 25 wt. %, in particular from 3 wt. % to 20 wt. %, and particularly preferably in amounts from 1 wt. % to 15 wt. %. Among these, the crystalline sodium aluminosilicates in detergent quality, in particular zeolite A, zeolite P and zeolite MAP, and optionally zeolite X, are preferred. Suitable aluminosilicates in particular comprise no particles having a particle size above 30 μm, and preferably have a content of at least 80 wt. % of particles having a size of less than 10 μm. The calcium-binding capacity is generally in the range of 100 to 200 mg CaO per gram.

In particular alkali carbonates, alkali hydrogen carbonates and/or sesquicarbonates are preferred water-soluble builder substances. In particular, sodium and/or potassium carbonates and hydrogen carbonates are used in the solid textile detergents. Optionally, it is also possible for lower amounts of calcium carbonates to be present in the solid textile detergents.

In addition or as an alternative to the described water-insoluble aluminosilicate and water-soluble alkali carbonate, further water-soluble inorganic builder materials may be present. These include in particular the water-soluble crystalline and/or amorphous alkalisilicate builders. Agents according to the invention preferably contain such water-soluble inorganic builder materials in amounts of 1 wt. % to 20 wt. %, in particular 5 wt. % to 15 wt. %. The alkali silicates that can be used as builder materials preferably have a molar ratio of alkali oxide to SiO₂ of less than 0.95, in particular of 1:1.1 to 1:12 and can be present in amorphous or crystalline form. Preferred alkali silicates are sodium silicates, in particular the amorphous sodium silicates, having a molar ratio of Na₂O:SiO₂ of 1:2 to 1:2.8. Crystalline silicates, which may be present either alone or in a mixture with amorphous silicates, that are used are preferably crystalline phyllosilicates of general formula Na₂SiₓO₂ₓ₊₁.yH₂O, where x, the so-called module, is a number from 1.9 to 4, and y is a number from 0 to 20, and preferred values for x are 2, 3 or 4. Preferred crystalline phyllosilicates are those in which x in the above-mentioned general formula takes on the value 2 or 3. In particular, both β- and δ-sodium disilicates (Na₂Si₂O₅.y H₂O) are preferred. Practically anhydrous crystalline alkali silicates, produced from amorphous alkali silicates, of the above general formula, in which x denotes a number from 1.9 to 2.1, can also be used in agents according to the invention. In a further preferred embodiment of agents according to the invention, a crystalline sodium phyllosilicate having a module from 2 to 3 is used, as it can be produced from sand and soda. Sodium silicates having a module in the range from 1.9 to 3.5 are used in a further embodiment of agents according to the invention. In a preferred embodiment of agents according to the invention, a granular additive made of alkali silicate and alkali carbonate is used.

Suitable graying inhibitors or active soil release ingredients are cellulose ethers, such as carboxymethyl cellulose, methyl cellulose, hydroxyalkyl celluloses and cellulose mixed ethers, such as methyl hydroxyethyl cellulose, methyl hydroxypropyl cellulose and methyl carboxymethylcellulose. Preferably, sodium carboxymethylcellulose, hydroxypropyl methyl cellulose, and the mixtures thereof, and optionally the mixtures thereof with methyl cellulose, are used. The active soil release ingredients that are customarily used include copolyesters containing dicarboxylic acid units, alkylene glycol units, and polyalkylene glycol units. The proportion of graying inhibitors and/or active soil release ingredients in the agents according to the invention generally does not exceed 2 wt. %, and preferably is 0.5 wt. % to 1.5 wt. %.

Derivatives of diaminostilbene disulfonic acid or the alkali metal salts thereof may be present, for example, as optical brighteners, in particular for textiles made of cellulose fibers (such as cotton). For example, salts of 4,4′-bis(2-anilino-4-morpholino-1,3,3.5-triazine-6-yl-amino)stilbene-2,2′-disulfonic acid, or similarly structured compounds carrying a diethanolamino group, a methylamino group or a 2-methoxyethylamino group instead of the morpholino group, are suitable. Moreover, brighteners of the type of the substituted 4,4′-distyryl diphenyl can be present, for example 4,4′-bis(4-chloro-3-sulfostyryl)biphenyl. It is also possible to use mixtures of brighteners. Brighteners of the type of the 1,3-diaryl-2-pyrazoline, for example 1-(p-sulfoamoylphenyl)-3-(p-chlorophenyl)-2-pyrazo ine and equivalently structured compounds are particularly well suited for polyamide fibers. The content of optical brighteners or brightener mixtures in the agent generally does not exceed 1 wt. %, preferably 0.05 wt. % to 0.5 wt. %. In a preferred embodiment of the invention, the agent is free of such active ingredients.

The customary foam regulators that can be used in the agents according to the invention include polysiloxane/silica mixtures, for example, wherein the finely divided silica contained therein is preferably silanized or hydrophobized in another manner. The polysiloxanes can be composed of either linear compounds or cross-linked polysiloxane resins and the mixtures thereof Further defoamers include paraffin hydrocarbons, in particular microparaffins and paraffin waxes, having a melting point above 40° C., saturated fatty acids or soaps including in particular 20 to 22 carbon atoms, for example sodium behenate, and alkali salts of monoalkyl and/or dialkyl esters of phosphoric acid, in which the alkyl chains each have 12 to 22 carbon atoms. Among these, the use of sodium monoalkyl phosphate and/or dialkyl phosphate having C₁₆ to C₁₈ alkyl groups is preferred. The proportion of foam regulators can preferably be 0.2 wt. % to 2 wt. %.

To set the desired pH value, the agents according to the invention can comprise systems and environmentally compatible acids, in particular citric acid, acetic acid, tartaric acid, malic acid, lactic acid, glycolic acid, succinic acid, glutaric acid and/or adipic acid, but also mineral acids, in particular sulfuric acid, alkali hydrogen sulfates, or bases, in particular ammonium or alkali hydroxides. Such pH regulators are preferably present in the agents according to the invention in amounts not above 10 wt. %, in particular 0.5 wt. % to 6 wt %.

The agents according to the invention are preferably present in the form of powdery, granular or tablet-shaped preparations, which can be produced in the manner known per se, for example by mixing, granulating, roller compacting, extruding and/or spray drying of the thermally resistant components, and admixing the more sensitive components, which include in particular enzymes, bleaching agents, and bleach-activating systems. The solid textile detergents can have a bulk density of 350 to 950 g/L, and post-compacted agents even higher than that. To produce agents according to the invention having increased bulk density, in particular in the range from 650 g/L to 950 g/L, a method comprising an extrusion step is preferred.

So as to produce textile detergents according to the invention in tablet form, the procedure is preferably such that all components or different additives are mixed with each other in a mixer, and the mixture is pressed by way of conventional tablet presses, such as eccentric presses or rotary tablet presses, using pressures in the range of 200·10⁵ Pa to 1500·10⁵ Pa. This readily yields break-resistant tablets that nonetheless dissolve sufficiently quickly under usage conditions, with flexural strength normally above 150 N. A tablet thus produced preferably has a weight of 15 g to 40 g, in particular of 20 g to 30 g, at a diameter of 35 mm to 40 mm.

The invention further relates to a method for cleaning textiles, wherein a solid textile detergent according to the invention is used in at least one method step. These include both manual and mechanical methods, wherein mechanical methods are preferred due to the precise controllability of the same, for example as far as the amounts used and application times are concerned. Methods for cleaning textiles are generally characterized in that, in one or more method steps, different substances with cleaning action are applied to the product to be cleaned and washed off following the application time, or that the product to be cleaned is treated in another manner with a detergent or a solution of this agent. All content, subject matter and embodiments that are described for textile detergents according to the invention can also be applied to this subject matter of the invention. Express reference is therefore made at this point to the disclosure provided elsewhere, noting that this disclosure also applies to the above-described methods according to the invention.

The advantages in the use of the solid textile detergents according to the invention over solid textile detergents comprising conventionally used proteases become apparent to the consumer in particular in methods that are carried out at a temperature between 10° C. and 50° C., preferably between 10° C. and 40° C., and particularly preferably between 20° C. and 40° C.

The proteases used in agents according to the invention can advantageously be used corresponding to the above comments in solid textile detergents and methods for laundering textiles. They can thus advantageously be used to provide a proteolytic activity in corresponding agents and methods.

The invention therefore further relates to the use of a protease
(a1) which includes an amino acid sequence that is at least 80% identical to the amino acid sequence indicated in SEQ ID NO. 1 and contains the amino acid glutamic acid (E) or aspartic acid (D) at position 99 in the count according to SEQ ID NO. 1; or
(a2) which includes an amino acid sequence that is at least 80% identical to the amino acid sequence indicated in SEQ ID NO. 1 and contains the amino acid asparagine (N) or glutamine (Q) at position 99 in the count according to SEQ ID NO. 1; or
(a3) which includes an amino acid sequence that is at least 80% identical to the amino acid sequence indicated in SEQ ID NO. 1 and contains the amino acid alanine (A) or glycine (G) or serine (S) at position 99 in the count according to SEQ ID NO. 1, so as to provide a proteolytic activity in a solid textile detergent having a pH value in the range from 4.0 to less than 10 in a 1 wt. % solution in deionized water at 20° C.

In a further embodiment, this use is characterized in that the protease, in the count according to SEQ ID NO. 1, furthermore includes at least one of the following amino acids:

(a) threonine at position 3 (3T);

(b) isoleucine at position 4 (4I);

(c) alanine, threonine or arginine at position 61 (61A, 61T or 61R);

(d) aspartic acid or glutamic acid at position 154 (154D or 154E);

(e) proline at position 188 (188P);

(f) methionine at position 193 (193M);

(g) isoleucine at position 199 (199I);

(h) aspartic acid, glutamic acid or glycine at position 211 (211D, 211E or 211G),

(i) combinations of amino acids (a) to (h).

All content, subject matter and embodiments that are described for the solid textile detergent can also be applied to the described uses. Express reference is therefore made at this point to the disclosure provided elsewhere, noting that this disclosure also applies to the above-described uses according to the invention.

It is also particularly advantageous to use a solid textile detergent according to the invention to remove protease-sensitive soiling on textiles. Particularly preferred embodiments are, for example, hand washing, manually removing stains from textiles, or the use in connection with a machine-based method.

### EXAMPLES

The product Persil Megaperls® Color, as it was commercially available in Germany in April 2013 and which contained a protease from the prior art that exhibited very good washing performance at temperatures of 40° and below, served as the comparison product V for the solid textile detergent E according to the invention, which with the exception of the protease was identical to the comparison product Persil Megaperls® Color. Instead of the conventional protease, a protease according to SEQ ID NO. 2 was used in the solid textile detergent E according to the invention, which at positions 1 to 98 and 100 to 269 agreed with SEQ ID NO. 2 and at position 99 in the count according to SEQ ID NO. 1 included the amino acid glutamic acid E. Both products E and V contained 0.76 wt. % of the respective solid formulated enzyme preparation forms (enzyme granules), based on the respective agent. The two products contained further enzymes in identical amounts. Both products E and V were tested for the performance thereof with respect to protease-sensitive soiling.

For this purpose, 3.5 kg of clean ballast load and 4 soil cloths, as they are commercially available, were each washed with 67.5 g of E and V. respectively, using a standard 40° C. program in a Miele washing machine and using water having a hardness of 15 to 17 ° d and subsequently dried (ABE test protocol). The following soil cloths were used:
CFT CS01—blood on cotton
CFT C03—chocolate milk/soot on cotton
EMPA 112—cocoa soiling on cotton
EMPA 163—porridge soiling on cotton

The degree of whiteness as a measure of cleaning performance, which is to say the lightening of soils, was measured by way of the spectrometer Minolta CM508d. The device had previously been calibrated to 100% using a supplied white standard.

Table 1 indicates the differences in the remission values (RD) for E over V (which is to say, the remission values of the soil cloths washed with E were higher by the difference value indicated in Table 1 than for the soil cloths washed with V) as mean values from 6 determinations and the errors in the 6-fold. determination (HSD).

TABLE 1
Washing results (difference in remission values RD)

| Soil cloth | RD (E compared to V) | HSD |
|---|---|---|
| CFT CS01 | 13.8 | 3.9 |
| CFT C03 | 7.9 | 1.7 |
| EMPA 112 | 4.8 | 1.5 |
| EMPA 163 | 3.4 | 2.1 |

All measured difference values are significant.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A solid textile detergent, comprising:
(a1) a protease including an amino acid sequence that is at least 80% identical to the amino acid sequence indicated in SEQ ID NO. 1 and contains the amino acid glutamic acid (E) or aspartic acid (D) at position 99 in the count according to SEQ ID NO. 1; or
(a2) a protease including an amino acid sequence that is at least 80% identical to the amino acid sequence indicated in SEQ ID NO. 1 and contains the amino acid asparagine (N) or glutamine (Q) at position 99 in the count according to SEQ ID NO. 1; or
(a3) a protease including an amino acid sequence that is at least 80% identical to the amino acid sequence indicated in SEQ ID NO. 1 and contains the amino acid alanine (A) or glycine (G) or serine (S) at position 99 in the count according to SEQ ID NO. 1,
wherein the solid textile detergent in a 1 wt. % solution in deionized water at 20° C. has a pH value in a range from 4.0 to less than 10.

2. The solid textile detergent according to claim 1, characterized in that the protease, in the count according to SEQ ID NO. 1, furthermore includes at least one of the following amino acids:
(a) threonine at position 3 (3T);
(b) isoleucine at position 4 (4I);
(c) alanine, threonine or arginine at position 61 (61A, 61T or 61R);
(d) aspartic acid or glutamic acid at position 154 (154D or 154E);
(e) proline at position 188 (188P);
(f) methionine at position 193 (193M);
(g) isoleucine at position 199 (199I);
(h) aspartic acid, glutamic acid or glycine at position 211 (211D, 211E or 211G),
(i) combinations of amino acids (a) to (h).

3. The solid textile detergent according to claim 1, comprising at least one additional enzyme of the same or of another type.

4. The solid textile detergent according to claim 1 wherein the at least one further enzyme comprises a protease, amylase, cellulase, hemicellulase, mannanase, tannase, xylanase, xanthanase, xyloglucanase, β-glucosidase, pectinase, carrageenase, perhydrolase, oxidase, oxidoreductase, lipase, or mixtures thereof.

5. The solid textile detergent according to claim 1 wherein the detergent contains no inorganic or organic peroxide source.

6. The solid textile detergent according to claim 1, characterized by having a pH value (1 wt. % solution in deionized water at 20° C.) of 6.5 to 9.5.

7. A method for cleaning textiles, comprising the step of contacting the textiles with a solid textile detergent according to claim 1.

8. The method according to claim 7, characterized by being carried out at a temperature between 10° C. and 50° C.


SEQUENCE LISTING (2 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR